(19) United States
(12) Patent Application Publication
(10) Pub. No.: US 2007/0272777 A1
(43) Pub. Date: Nov. 29, 2007

(54) Processes for reducing particle size of aripiprazole

(76) Inventors: Guy Samburski, Ganot-Hadar (IL); Hagit Eisen-Nevo, Shoam (IL); Judith Aronhime, Rehovot (IL); Sigalit Levi, Modi'in (IL)

Correspondence Address: KENYON & KENYON LLP, ONE BROADWAY, NEW YORK, NY, 10004, US

(21) Appl. No.: 11/644,567

(22) Filed: Dec. 22, 2006

Related U.S. Application Data
(60) Provisional application No. 60/753,466, filed on Dec. 22, 2005.

(51) Int. Cl. B02C 19/08; C07D 401/12

(52) U.S. Cl. 241/30; 544/363

(57) ABSTRACT

Micronized aripiprazole Form II having a particle shape and wherein 90% or more of the particles have a particle size of about 30 μm and the micronized aripiprazole Form II does not have more than 10% by weight of aripiprazole Type C as determined by X-ray diffraction and processes for making thereof.

### RELATED APPLICATIONS

This application claims the benefit of U.S. provisional Application No. 60/752,466, filed Dec. 22, 2005 hereby incorporated by reference.

### FIELD OF THE INVENTION

The present invention encompasses processes for reducing the particle size of aripiprazole Form II wherein no more than about 10% by weight of the resulting aripiprazole is transformed into aripiprazole Type C.

### BACKGROUND OF THE INVENTION

Schizophrenia is the most common type of psychosis, caused by excessive neurotransmission activity of the dopaminergic nervous system in the central nervous system. A number of drugs which block the neurotransmission of dopaminergic receptor in the central nervous system have been developed for use in treating schizophrenia.

Among the drugs developed are phenothiazine-type compounds such as chlorpromazine, butyrophenone-type compounds such as haloperidol, and benzamide-type compounds such as sulpiride. These drugs improve so-called positive symptoms in the acute period of schizophrenia such as hallucinations, delusions, and excitations. However, these drugs are not effective for improving the so-called negative symptoms which are observed in the chronic period of schizophrenia such as apathy, emotional depression, and hypopsychosis. The drugs currently used produce undesirable side effects such as akathisia, dystonia, Parkinsonism dyskinesia, and late dyskinesia, by blocking the neurotransmission of dopaminergic receptor in the striate body. Drugs that improve both the negative and positive symptoms of schizophrenia but diminish the undesirable side effect of schizophrenia are particularly desirable.

Aripiprazole, of the formula 7-{4-[4-(2,3-dichlorophenyl)-1-piperazinyl]-butoxy}-3,4-dihydro carbostyril or 7-{4-[4-(2,3-dichlorophenyl)-1-piperazinyl]-butoxy}-3,4-dihydro-2(1H)-quinolinone, has a molecular weight of 448.38 and the following structure:

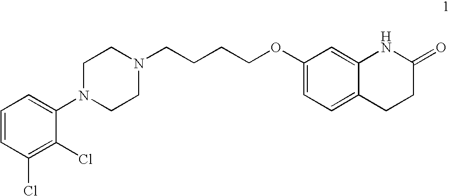

This molecule is an atypical antipsychotic agent, useful in treating schizophrenia, marketed under the name Abilify® by Bristol-Myers Squibb. Its therapeutic uses were disclosed in U.S. Pat. Nos. 4,734,416 and 5,006,528. This pyschotropic drug exhibits high affinity for dopamine D₂ and D₃, serotonin 5-HT_{1A} and 5-HT_{2A} receptors; moderate affinity for dopamine D₄, serotonin 5-HT_{2C} and 5-HT₇, α₁-adrenergic and histamine H₁ receptors; and moderate affinity for the serotonin reuptake site. Aripiprazole has no appreciable affinity for cholinergic muscarinic receptors. It has been proposed, that the effectiveness of Aripiprazole is mediated through a combination of partial agonist activity at D₂ and 5-HT_{1A} receptors as well as antagonist activity at 5-HT_{2A} receptors.

Japanese Patent Kokai No. 02-191256 discloses that anhydride crystals of aripiprazole are typically manufactured by recrystallization of aripiprazole from ethanol or by heating aripiprazole hydrate at a temperature of 80° C. According to WO 03/26659, anhydride aripiprazole prepared by these methods is significantly hygroscopic.

The Proceedings of the 4^{th} Japanese-Korean Symposium on Separation Technology (Oct. 6-8, 1996) disclosed that aripiprazole anhydride crystals may exist as Type-I and Type-II crystals. Type-I aripiprazole crystals can be prepared by recrystallizing aripiprazole from an ethanol solution or by heating aripiprazole hydrate at 80° C. Type-II aripiprazole crystals can be prepared by heating the Type-I crystals at 130° C. to 140° C. for 15 hours. This process is not easily applied to an industrial scale preparation of anhydride aripiprazole.

PCT publication WO 03/26659 apparently discloses the preparation of anhydrous aripiprazole Type A, B, C, D, E, F and G and aripiprazole hydrate A. Anhydrous aripiprazole Type C (herein “Type C”) is seemingly characterized by a powder X-ray diffraction spectrum having characteristic peaks at 2θ=12.6°, 13.7°, 15.4°, 18.1°, 19.0°, 20.6°, 23.5° and 26.4°.

PCT publication WO 05/058835 discloses anhydrous aripiprazole crystalline forms, among them Form II, characterized by X-ray powder diffraction peaks at about 16.5, 18.7, 21.9, 22.4, and 23.5 degrees two-theta, +0.2 degrees two-theta. Further disclosed is substantially pure Form II, having less than 40% of other aripiprazole crystalline forms and more preferably no more than 10% by weight of other aripiprazole crystalline forms, including Type C. In addition, it is disclosed that heating of Form II results in transformation to Type C.

An important process necessary for drug formulation involves grinding, crushing and milling as a means for reducing particle size distribution in an effort to improve formulation homogeneity or dissolution and bioavailability. In the case of aripiprazole Form II, it is especially important to develop methods for milling, that maintain polymorphic integrity, because unwanted polymorphic transformation can lead to difficulties during formulation and storage. Accordingly, there is a need in the art for processes for reducing particle size of aripiprazole Form II which minimizes or eliminates the amount of polymorphic transformation in the resulting aripiprazole.

### SUMMARY OF THE INVENTION

One embodiment of the invention encompasses micronized aripiprazole Form II having a particle shape and wherein 90% or more of the particles have a particle size of about 30 μm and the micronized aripiprazole Form II does not have more than 10% by weight of aripiprazole Type C as determined by X-ray diffraction.

Another embodiment of the invention encompasses a method of preparing micronized aripiprazole Form II, by wherein the micronized aripiprazole Form II does not have aripiprazole Type C in an amount of not more than 10% by weight as determined by X-ray diffraction and wherein not less than 90% of the micronized aripiprazole has a particle size of about 30 μm.

Yet another embodiment of the invention encompasses a process for reducing aripiprazole Form II particle size comprising providing aripiprazole Form II; and reducing the particle size of aripiprazole Form II to yield particulate aripiprazole Form II, wherein the amount of aripiprazole Type C in the particulate aripiprazole Form II is not more than 10% by weight as determined by X-ray diffraction of the amount of aripiprazole Type C in the aripiprazole Form II, provided that particle size reduction is not performed with a ball mill. Preferably, the amount of aripiprazole Type C in the aripiprazole Form II is not more than 5% by weight as determined by X-ray diffraction and more preferably, not more than 1% by weight.

Preferably, reduction of the particle size of aripiprazole Form II is performed with a cone mill, mortar and pestle, or micronizer.

Another embodiment of the invention encompasses pharmaceutical compositions comprising the micronized aripiprazole Form II of the present invention and one or more pharmaceutically acceptable excipients, diluents or carriers.

Another embodiment of the invention encompasses pharmaceutical compositions comprising the micronized aripiprazole Form II obtained by the process of the invention and one or more pharmaceutically acceptable excipients, diluents or carriers.

### BRIEF DESCRIPTION OF THE DRAWINGS

FIG. 1 illustrates the X-ray diffraction, before and after milling of aripiprazole Form II by conical mill as described in Example 1.

FIG. 2 illustrates the X-ray diffraction, before and after grinding of aripiprazole Form II by mortar and pestle as described in Example 2.

FIG. 3 illustrates the X-ray diffraction, before and after grinding of aripiprazole Form II by Ball mill as described in Example 5

FIG. 4 illustrates the X-ray diffraction, before and after grinding of frozen aripiprazole Form II by cone mill as described in Example 4.

FIG. 5 illustrates the X-ray diffraction, before and after grinding of aripiprazole Form II by Micronizer as described in Example 3c.

FIG. 6A illustrates the X-ray diffraction for Form II having 5%, 10%, 20% and 30% of Type C. The characteristic peak of Type C at 20.8 is marked by an arrow.

FIG. 6B illustrates the X-ray diffraction for Form II having 5%, 10%, 20% and 30% of Type C with focus on the area between 13 and 29 degrees. The ratio between the height of Form II peak (at 20.4 degrees) and between the heights of Type C peak (at 20.8 degrees) is calculated for the mixtures.

### DETAILED DESCRIPTION OF THE INVENTION

As discussed above, an important process necessary for drug formulation involves reducing solid material particle size to a desired size. Devices which perform this function include: ball or media mills, cone and gyratory crushers, jet and fluid energy mills, etc. Because reducing the particle size of aripiprazole can either result in aripiprazole polymorphic conversion or in the generation of an amorphous aripiprazole, and because Form II is known to result in transformation to Type C upon heating, discovery of methods for reducing average particle size of aripiprazole Form II where there is no polymorphic conversion, is desired. For instance, when using the ball-mill method to create particles of aripiprazole Form II, the aripiprazole Form II was converted into aripiprazole Type C. Example 5 illustrates this undesired conversion. These conversions of one crystal form into another crystal form during milling are not desired since they affect the physicochemical, formulation and processing parameters of the aripiprazole. A mixture of various polymorphs may have variable properties which are unacceptable in view of stringent GMP requirements, which demands consistency among batches. The processes of the invention yield a stable aripiprazole Form II particle without significant transformation to Type C, thereby achieving GMP requirements such as consistency among batches.

The present invention encompasses aripiprazole Form II, wherein the amount of aripiprazole Type C in the particulate aripiprazole Form II is present in not more than 10% by weight as determined by X-ray diffraction and wherein not less than 90% of the particles have a particle size of about 30 μm. Preferably, not less than 90% of the particles have a particle size of about 20 μm, and more preferably of about 10 μm. Preferably, the aripiprazole Type C is present in not more than 5% by weight as determined by X-ray diffraction, and more preferably not more than 1%.

The present invention additionally encompasses a method of preparing bulk aripiprazole, wherein not less than 90% of the particles have a particle size of about 30 μm and wherein the amount of aripiprazole Type C in the particulate aripiprazole Form II is not more than 10% by weight as determined by X-ray diffraction.

The method for micronizing aripiprazole Form II comprises providing aripiprazole Form II; and milling the aripiprazole Form II using a cone mill, mortar and pestle, or micronizer to yield particulate aripiprazole Form II, wherein the amount of aripiprazole Type C in the particulate aripiprazole Form II is not more than 10% by weight as determined by X-ray diffraction from the amount of aripiprazole Type C in the aripiprazole Form II.

The milling step may be achieved by grinding with a conical mill using a micron screen in the size of about 470 micron screen to 830 micron screen. When using a conical mill, the milling speed is about 1315 rpm to about 4710 rpm. Preferably, when using a 470 micron screen the milling speed is about 4710 rpm. Preferably, when using a 830 micron screen the milling speed is about 1315 rpm.

Alternatively, the milling step may be achieved by vigorous hand grinding with a mortar and pestle for about 0.5 min. to 10 min. and preferably for about 1 min.

Alternatively, the milling step may be achieved by grinding with a micronizer wherein the micronizer air settings are feeding air ranging from about 2.0 bars to about 6.0 bars and grinding air range of about 1.0 to about 5.0 bars. The milling step may be performed with a conical mill using a micron screen in the size of about 470 micron screen to 830 micron screen and a milling speed of about 1315 rpm to about 4710 rpm. Preferably, the milling step is performed with a conical mill using a 470 micron screen and a milling speed is about 4710 rpm and more preferably, the milling step is performed with a conical mill using a 830 micron screen and a milling speed of about 1315 rpm. When the milling step is performed by grinding with a micronizer, the micronizer air settings are feeding air of about 2.0 bars to about 6.0 bars and grinding air range of about 1.0 to about 5.0 bars.

The starting aripiprazole Form II of the process can be made using the process described in PCT publication WO 05/058835 or U.S. application Ser. No. 11/015,068 filed Dec. 16, 2004, hereby incorporated by reference.

Preferably, the amount of aripiprazole Type C in the particulate aripiprazole Form II is not more than 5% by weight as determined by X-ray diffraction. More preferably, the amount of aripiprazole Type C in the particulate aripiprazole Form II is not more than 1% by weight as determined by X-ray diffraction. Preferably, the starting aripiprazole Form II is frozen to achieve an increase of less than 1% by weight of aripiprazole Type C.

Preferably, not less than 90% of the particles have a particle size of about 20 μm, and more preferably of about 10 μm.

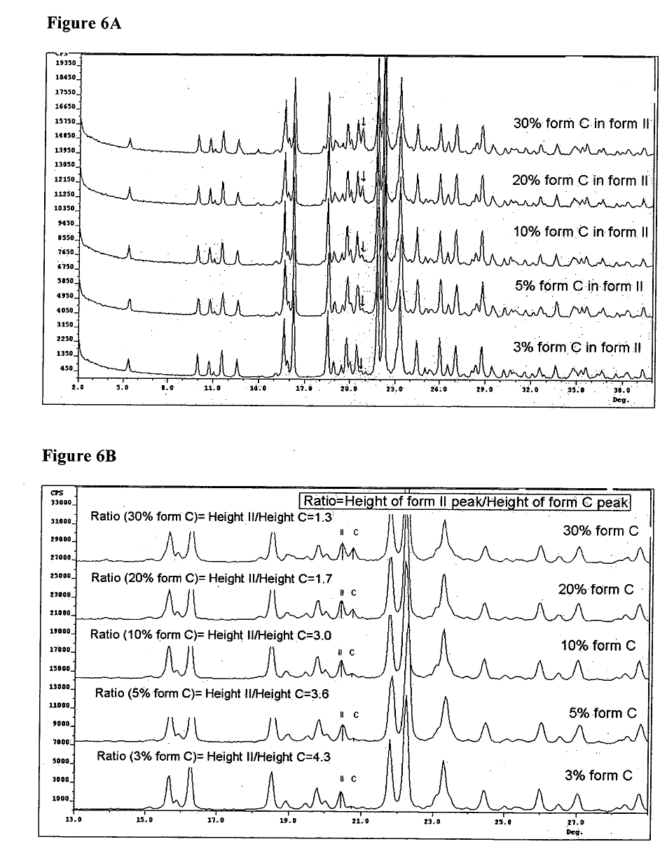

The amount of Type C in Form II is determined by comparing the peak height ratio between a peak for aripiprazole Form II and aripiprazole Type C to a standard. The peak for aripiprazole Form II is at about 20.4° 2-theta and the peak for aripiprazole Type C is at about 20.8° 2-theta. Prior to grinding, to determine the initial amount of aripiprazole Type C the peak height ratio is compared to a standard. Similarly, after grinding, to determine the final amount of aripiprazole Type C, the peak height ratio again is compared to a standard. Thus, each step quantifies the amount of aripiprazole Type C in aripiprazole Form II. FIG. 6B illustrates this method. Also see, Polymorphism in Molecular Crystals, Joel Bernstein, pp. 117-125 (Oxford Science Publications, 2002). The standard may be prepared by mixing specific amounts of Type C and Form II and determining the X-ray diffraction pattern.

This method is also illustrated in FIGS. 6A and B which are used to calculate the ratio between the heights of the peaks in the known mixtures. Mixtures of aripiprazole Form II with 3%, 5%, 10%, 20% and 30% of aripiprazole Type C are used as a standard to convert height ratio to weight % of Type C in the sample. Determining the percentage increase in aripiprazole Type C in the samples is achieved by calculating the percent of Type C in the XRD diffractogram of the sample before and after grinding.

Having thus described the invention with reference to particular preferred embodiments and the following illustrative examples, those in the art would appreciate modifications to the invention as describes and illustrated that do not depart from the spirit and scope of the invention as disclosed in the specification. The examples are set forth to aid in understanding the invention but are not intended to, and should not be construed to, limit its scope in any way. The examples do not include detailed descriptions of conventional methods. Such methods are well known to those of ordinal skill in the art and are described in numerous publications.

### EXAMPLES

### Instrumentation

X-Ray powder diffraction data are obtained by using methods known in the art such as using a SCINTAG powder X-Ray diffractometer model X'TRA equipped with a solid-state detector using a copper radiation of 1.5418 Å. A round aluminum sample holder with zero background was used. The scanning parameters included: range: 2-40 degrees two-theta; scan mode: continuous scan; step size: 0.05 deg.; and a rate of 3 deg/min. Running in spin/oscillate mode. All peak positions are reported with an error of ±0.2 degrees two theta.

### Example 1

### Milling by Cone Mill

A) Aripiprazole Form II (100 g) was milled in a conical mill (Quadro comil 197) using a 470 micron screen and a milling speed of 4710 rpm. The milled sample was analyzed by XRD and found to contain less than 5% Type C.

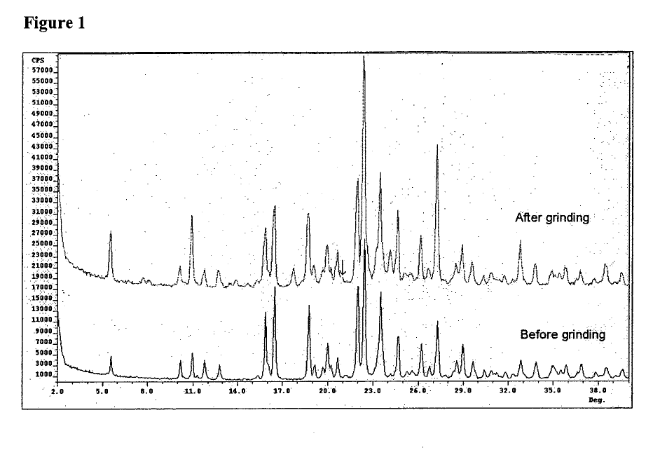

B) Aripiprazole Form II (100 g) was milled by using a 830 microns screen and a milling speed of 1315 rpm. The milled samples were analyzed by XRD. The ratio of the peak height at 20.4° 2-theta (Form II) and the peak height at 20.8° 2-theta (Type C) was about 5. Comparing the ratio of 5 to the standard shown in 6b indicated that less than 5% by weight of Type C was present. (See FIG. 1).

### Example 2

### Grinding by Mortar and Pestle

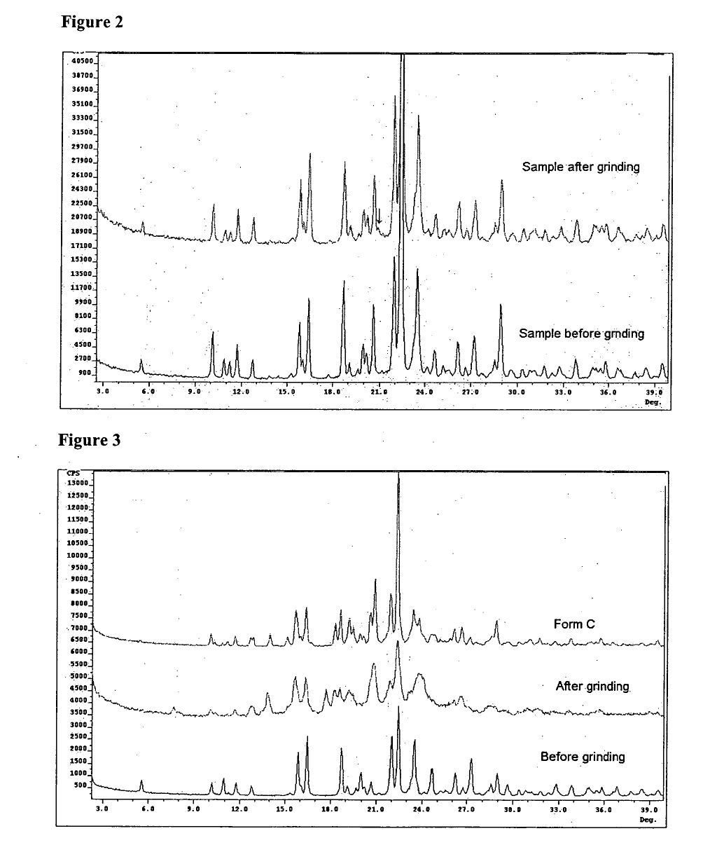

Aripiprazole Form II (200 mg) was ground vigorously using mortar and pestle for about 1 min. The sample was analyzed by XRD. Comparing the XRD diffractogram of the sample before grinding to XRD diffractogram of the sample after grinding by mortar and pestle showed a small characteristic peak of Type_C at 20.8° 2-theta. See FIG. 2, the marked arrow. The ratio between the height of the peak at 20.4° 2-theta (Form II) and the height of the peak at 20.8° 2-theta (Type_C) was about 6. Thus, using the standard shown in FIG. 6b, the ratio of 6 indicated that the milled sample contained about less than 5% Type C.

### Example 3

### Grinding by Micronizer

A) Aripiprazole Form II (100 g) was micronized using a micronizer (Micronizer Sturtevant 50 mm) with settings at Feeding Air of 5.0 bars and Grinding Air of 4.0 bars. According to the XRD, the milled sample did not contain Type_C.

B) Aripiprazole Form II (100 g) was micronized using a micronizer (Micronizer Sturtevant 50 mm) with settings at Feeding Air of 3.0 bars and Grinding Air of 2.0 bars. According to the XRD, the milled sample did not contain Type_C.

C) Aripiprazole Form II (4 kg) was micronized using a micronizer (Micronizer Sturtevant 50 mm) with settings at Feeding Air of 6.0 bars and Grinding Air of 5.0 bars. The sample was analyzed by XRD (FIG. 5). According to the XRD, the milled sample did not contain Type_C.

The XRD diffractogram of Form II milled by micronizer indicated that the sample remained as Form II (see FIG. 5). The fact that the characteristic peak of Type C at about 20.8° 2-theta was not detected showed that Form II did not transform significantly to Type C when Form II was milled using a micronizer.

### Example 4

### Milling by Cone Mill of Frozen Aripiprazole Form II

A) Aripiprazole Form II (100 g) was stored at a freezing temperature of −10° C. to −20° C. for a period of 24 hours until frozen. The frozen material was milled in a conical mill (Quadro comil 197) using a 470 microns screen and milling speed of 4710.

B) Aripiprazole Form II (100 g) was stored at a freezing temperature of −10° C. to −20° C. for a period of 24 hours until frozen. The frozen material was milled in a conical mill using a 830 microns screen and milling speed of 1315 rpm.

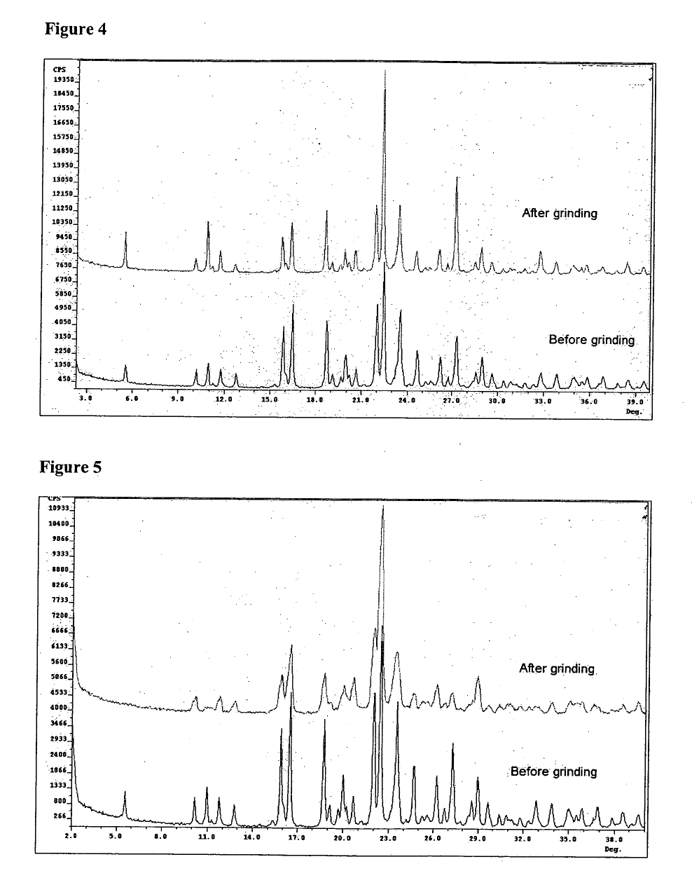

In both cases, the XRD diffractogram of the milled form was identical to the XRD diffractogram of the sample before the milling. See FIG. 4. The characteristic peak of Type C at about 20.8° 2-theta was not detected in the diffractogram of the milled sample showing that the aripiprazole Form II was not transformed to Type C by this method to any significant measure.

### Example 5

### Comparative Example

### Using Ball Mill

Aripiprazole Form II (100 g) was milled in a centrifugal ball mill (Retch S-100), operated for 90 minutes at 580 rpm. The milling media was 26 stainless steel balls in a 250 mm stainless steel jar. The sample was analyzed by XRD. A comparison of the XRD diffractogram before and after milling the sample showed a stark increase in the content of Type C. See FIG. 3. After grinding, the XRD diffractogram contained broad peaks, which was indicative that the crystallinity of the sample decreased. Accordingly, the ball-mill procedure cannot be used with aripiprazole where excessive polymorphic transformation is not desired.

What is claimed is:

1. A process for reducing aripiprazole Form II particle size comprising
providing aripiprazole Form II; and
milling the aripiprazole Form II using a cone mill, mortar and pestle, or micronizer to yield particulate aripiprazole Form II,
wherein the amount of aripiprazole Type C in the particulate aripiprazole Form II is not more than 10% by weight as determined by X-ray diffraction from the amount of aripiprazole Type C in the aripiprazole Form II.

2. The process according to claim 1, wherein the amount of aripiprazole Type C in the particulate aripiprazole Form II is not more than 5% by weight as determined by X-ray diffraction.

3. The process according to claim 1, wherein the amount of aripiprazole Type C in the particulate aripiprazole Form II is not more than 1% by weight as determined by X-ray diffraction.

4. The process according to claim 1, wherein the particulate aripiprazole Form II has a particle size of about 30 μm.

5. The process according to claim 1, wherein the particulate aripiprazole Form II has a particle size of about 20 μm.

6. The process according to claim 1, wherein the milling step is performed with a conical mill using a micron screen in the size of about 470 micron screen to 830 micron screen and a milling speed of about 1315 rpm to about 4710 rpm.

7. The process according to claim 6, wherein the milling step is performed with a conical mill using a 470 micron screen and a milling speed is about 4710 rpm.

8. The process according to claim 6, wherein the milling step is performed with a conical mill using a 830 micron screen and a milling speed of about 1315 rpm.

9. The process according to claim 1, wherein the milling step is performed by grinding with a micronizer, wherein the micronizer air settings are feeding air of about 2.0 bars to about 6.0 bars and grinding air range of about 1.0 to about 5.0 bars.

10. The process according to claim 1, wherein the amount of aripiprazole Type C in the particulate aripiprazole Form II is determined by comparing the height peak ratio of aripiprazole Form II at 20.4° 2-theta and the peak for aripiprazole Type C at 20.8° 2-theta and comparing the ratio to FIG. 6B.

11. Micronized aripiprazole Form II having a particle shape and wherein 90% or more of the particles have a particle size of about 30 μm and the micronized aripiprazole Form II does not have more than 10% by weight of aripiprazole Type C as determined by X-ray diffraction.

12. The micronized aripiprazole Form II of claim 11, wherein the amount of aripiprazole Type C in the particulate aripiprazole Form II is present in not more than 5% by weight as determined by X-ray diffraction.

13. The micronized aripiprazole Form II of claim 11, wherein the amount of aripiprazole Type C in the particulate aripiprazole Form II is present in not more than 1% by weight as determined by X-ray diffraction.

14. The micronized aripiprazole Form II of claim 11, wherein the particle size is about 20 μm.

15. A pharmaceutical composition comprising micronized aripiprazole Form II having a particle shape and wherein 90% or more of the particles have a particle size of about 30 μm and the micronized aripiprazole Form II does not have more than 10% by weight of aripiprazole Type C as determined by X-ray diffraction and at least one pharmaceutically acceptable excipient.

16. The pharmaceutical formulation according to claim 15, wherein the amount of aripiprazole Type C in the particulate aripiprazole Form II is present in not more than 5% by weight as determined by X-ray diffraction.

17. The pharmaceutical formulation according to claim 15, wherein the amount of aripiprazole Type C in the particulate aripiprazole Form II is present in not more than 1% by weight as determined by X-ray diffraction.

18. The pharmaceutical formulation according to claim 15, wherein the particle size is about 20 μm.
